(19) United States
(12) Patent Application Publication
(10) Pub. No.: US 2025/0205076 A1
(43) Pub. Date: Jun. 26, 2025

(54) FLUID COLLECTION DEVICES, RELATED SYSTEMS, AND RELATED METHODS

(71) Applicant: PUREWICK CORPORATION, Covington, GA (US)

(72) Inventor: Mark Spector, Loganville, GA (US)

(21) Appl. No.: 19/078,602

(22) Filed: Mar. 13, 2025

Related U.S. Application Data
(63) Continuation of application No. 17/051,585, filed on Oct. 29, 2020, now Pat. No. 12,274,638.
(60) Provisional application No. 62/665,297, filed on May 1, 2018.

(51) Int. Cl. A61F 5/44; A61F 5/453; A61F 5/455

(52) U.S. Cl. CPC A61F 5/4405; A61F 5/453; A61F 5/455; A61M 2202/0496; A61M 2210/1092; A61M 2210/1096

(57) ABSTRACT

In an example, a fluid collection device includes a fluid impermeable barrier that at least partially defines a chamber. The fluid impermeable barrier also defines an opening extending therethrough. The opening is configured to be positioned adjacent to a female urethra or have a male urethra positioned therethrough. The fluid collection device also includes a conduit at least partially disposed in the chamber. The conduit defines at least one aperture that allows an interior of the conduit to be in fluid communication with the chamber. The fluid collection device further includes a valve. The valve includes a first aperture coupled to the conduit and a second aperture that is fluid couplable to at least one of an air source, a vacuum source, or a fluid storage container. The valve is configured to control fluid flow through the conduit responsive to direction from a user.

### CROSS-REFERENCE TO RELATED APPLICATIONS

This application is a continuation of U.S. application Ser. No. 17/051,585 filed on Oct. 29, 2020, which is a U.S. National Stage application of PCT International Application No. PCT/US2019/029611 filed on Apr. 29, 2019, which claims priority to U.S. Provisional Application No. 62/665,297 filed on May 1, 2018, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pan can be prone to discomfort, spills, and other hygiene issues and urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are related to devices, systems, and methods of using fluid collection devices. In an embodiment, a fluid collection device includes a fluid impermeable barrier that at least partially defines a chamber. The fluid impermeable barrier also defines an opening extending therethrough. The opening is configured to be positioned adjacent to a female urethra or have a male urethra positioned therethrough. The fluid collection device also includes a conduit at least partially disposed in the chamber. The conduit defines at least one aperture that allows an interior of the conduit to be in fluid communication with the chamber. The fluid collection device further includes a valve. The valve includes a first aperture coupled to the conduit and a second aperture that is fluid couplable to at least one of an air source, a vacuum source, or a fluid storage container. The valve is configured to selectively control gas flow through the conduit responsive to actuation of the valve.

In an embodiment, a system to collect and transport fluid is disclosed. The system includes a vacuum source or a gas source. The system also includes a fluid storage container configured to collect a fluid. The fluid storage container is in fluid communication with the vacuum source or the air source. Additionally, the system includes a fluid collection device positioned upstream from the fluid storage container. The fluid collection device includes a fluid impermeable barrier at least partially defining a chamber. The fluid impermeable barrier also defines an opening extending therethrough. The opening is configured to be positioned adjacent to a female urethra or have a male urethra positioned therethrough. The fluid collection device also includes a conduit at least partially disposed in the chamber. The conduit defines at least one aperture therein that allows an interior of the conduit to be in fluid communication with the chamber. The fluid collection device further includes a valve including a first aperture coupled to the conduit and a second aperture that is configured to be in fluid communication with at least one of the gas source, the vacuum source, or the fluid storage container. The valve is configured to selectively control fluid flow through the conduit responsive to actuation of the valve.

In an embodiment, a method to collect fluid is disclosed. The method includes positioning an opening of a fluid collection device adjacent to a female urethra or around a male urethra. The opening is defined by a fluid impermeable barrier of the fluid collection device. The method also includes receiving fluid from the female urethra or the male urethra into a chamber of the fluid collection device, the chamber of the fluid collection device at least partially defined by the fluid impermeable barrier. Additionally, the method includes switching a valve between a first state and a second state. The valve is in fluid communication with a conduit that is at least partially disposed in the chamber. The conduit defines at least one aperture that allows an interior of the conduit to be in fluid communication with the chamber. The valve permits a flow of a gas through the conduit when the valve is in the first state and restricts the flow of the fluid of the fluid through the conduit when the valve is in the second state.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a schematic cross-sectional view of a fluid collection device according to an embodiment.

FIG. 2A is a schematic cross-sectional view of a fluid collection device, according to an embodiment.

FIGS. 2B and 2C are schematic cross-sectional views of the valve in first and second states, respectively, according to an embodiment.

FIG. 3 is a schematic cross-sectional view of a fluid collection device, according to an embodiment.

FIG. 4A is a schematic cross-sectional view of a fluid collection device, according to an embodiment.

FIG. 4B is a schematic cross-sectional view of a fluid collection device, according to an embodiment.

FIG. 5 is a schematic cross-sectional view of male fluid collection device, according to an embodiment.

FIG. 6 is a schematic illustration of a fluid collection system, according to an embodiment.

FIG. 7 is a schematic cross-sectional view of a fluid collection device that is configured to be coupled to a gas source, according to an embodiment.

FIG. 8 is a schematic of a fluid collection system, according to an embodiment.

FIG. 9 is a flow diagram of a method to use any of the fluid collection devices and/or fluid collection systems disclosed herein, according to an embodiment.

### DETAILED DESCRIPTION

Embodiments disclosed herein are related to devices, systems, and methods of using fluid collection devices. In an example, a fluid collection device includes a fluid impermeable barrier that at least partially defines a chamber. The fluid impermeable barrier also defines an opening extending therethrough. The opening is configured to be positioned adjacent to a female urethra or have a male urethra positioned therethrough. The fluid collection device also includes a conduit at least partially disposed in the chamber. The conduit defines at least one aperture that allows an interior of the conduit to be in fluid communication (e.g., fluidly coupled) with the chamber. The fluid collection device further includes a valve. The valve includes a first aperture coupled to the conduit and a second aperture that configured to be in fluid communication, directly (e.g., directly attached to or at least partially disposed in) and/or indirectly (e.g., via at least one tube), to at least one of an air source, a vacuum source, or a fluid storage container. The valve is configured to control fluid flow through the conduit responsive to direction from a user.

The fluid collection devices disclose herein are configured to collect fluids from an individual. The fluids collected by the fluid collection devices include urine. The fluids collected by the fluid collection devices can also include vagina discharge, penile discharge, reproductive fluids, blood, sweat, or other bodily fluids.

The conduit can be in indirect fluid communication to a gas source or a vacuum source, for example, via at least one tube or the valve. The gas source causes at least a gas (e.g., atmospheric gas, oxygen, etc.) to flow away from the gas source and through the interior of the conduit. The vacuum source causes at least a gas to flow towards the vacuum source and through the interior of the conduit. In an embodiment, the gas source or the vacuum source is configured to continuously flow a gas through the interior of the conduit. The continuous flow of the gas through the interior of the conduit can provide discomfort to an individual that uses the fluid collection device (e.g., the fluid collection device positioned adjacent to or around the individual's urethra). In an example, the continuous flow of the gas through the interior of the conduit can cause a gas to flow around the urethra of the individual thereby cooling the urethra of the individual. In an example, the continuous flow of the gas through the interior can cause a vacuum to be applied to portions of the individual that are proximate to the fluid collection device, thereby causing capillaries of the individual to burst (e.g., form a hickey). As such, it can be beneficial to limit the gas flow through the interior of the chamber to only when a fluid needs to be collected from the individual.

However, in some embodiments, it can be difficult to stop the continuous gas flow through the interior of the conduit. For example, the gas source or the vacuum source can be spaced from the individual using the fluid collection device. It can be difficult for the individual using the fluid collection device to turn the gas source or the vacuum source on or off since the individual may have limited mobility. In another example, the gas source or the vacuum source can be coupled to a network of tubes that allows the gas source or the vacuum source to supply the gas or vacuum to a variety of locations. In such an example, it can be impractical to turn the gas source or the vacuum source on or off merely for the comfort of a single individual since the other locations may need to use the gas source or the vacuum source.

The valve of the urine collection device improves the comfort of the individual using the fluid collection device because it allows the individual to control the gas flow (e.g., flow of atmospheric air, oxygen, nitrogen, etc.) through the conduit. For example, the valve can be switchable between a first state and a second state. The valve at least partially (e.g., completely) restricts and at least partially allows the gas flow through the conduit when the valve is in the first state and the second state, respectively. In other words, the valve allows more gas flow through the interior of the conduit when the valve is in the second state than when the valve is in the first state. The individual using the fluid collection device can manipulate an actuator that is coupled to or integrally formed with the valve thereby selectively and controllably switching the valve between the first and second states depending on the needs of the individual. For example, the individual can manipulate the actuator to switch the valve from the first state to the second state when the individual is about to urinate or otherwise requires fluids to be removed from the individual. In an embodiment, after the fluid collection device has collected at least some of the fluids, the individual can manipulate the actuator thereby switching the valve from the second state to the first state thereby increasing the comfort of the individual using the fluid collection device.

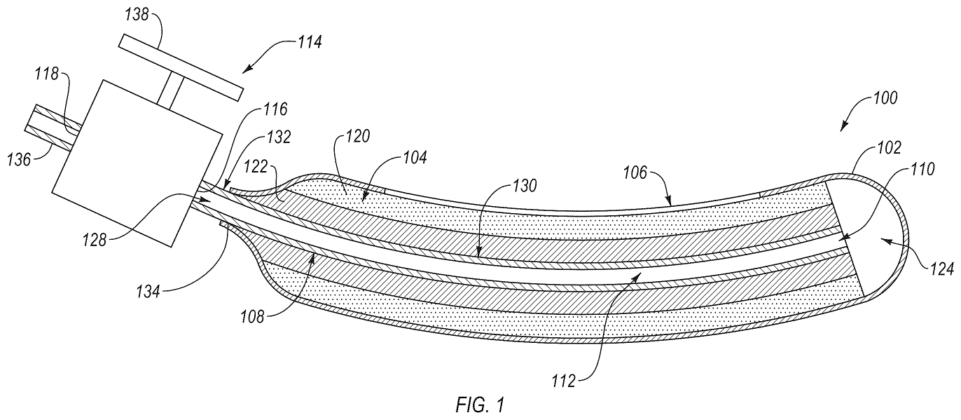

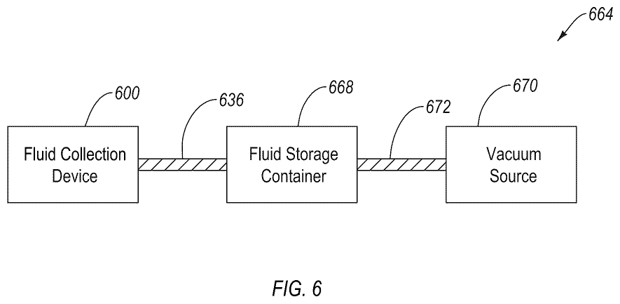

FIG. 1 is a schematic cross-sectional view of a fluid collection device 100 according to an embodiment. The fluid collection device 100 is an example of a female fluid collection device 100 that is configured to receive fluids from a female. The fluid collection device 100 includes a fluid impermeable barrier 102. The fluid impermeable barrier 102 at least partially defines a chamber 104 and an opening 106. The opening 106 extends through the fluid impermeable barrier 102 thereby enabling fluids to enter the chamber 104. The opening 106 can be configured to be positioned adjacent to a female urethra. The fluid collection device 100 also includes a conduit 108 (e.g., tube) that is at least partially disposed in the chamber 104. The conduit 108 defines at least one aperture 110 therein that allows an interior 112 of the conduit 108 to be in fluid communication with the chamber 104. In some examples, the conduit 108 may include one or more portions that are resilient, such as to have one or more of a diameter or wall thickness that allows the conduit to be flexible. In some examples, the conduit 108 may be frosted or opaque (e.g., black) to obscure the visibility of the fluids therein. The fluid collection device 100 also includes a valve 114 that is proximate to and spaced from the chamber 104. The valve 114 is positioned downstream from the aperture 110. The valve 114 is configured to selectively control the gas flow through the conduit 108 responsive to direction from an individual (e.g., responsive to an individual manipulating an actuator 138). For example, the valve 114 can control a gas flow through the interior 112 of the conduit 108 which, in turn, can control the flow of fluids through the conduit 108. The valve 114 includes a first aperture 116 that is couplable to (e.g., fluidly coupled and/or attachable to) the conduit 108. The valve 114 also includes a second aperture 118 that is configured to be in fluid communication, directly and/or indirectly to at least one of a vacuum source (e.g., vacuum source 670 of FIG. 6) or a fluid storage container (e.g., fluid storage container 668 of FIG. 6). For example, the second aperture 118 of the valve 114 may be in fluid communication with at least one of the vacuum source, or the fluid storage container.

The fluid collection device 100 is configured to receive the fluids into the chamber 104 via the opening 106. For example, the opening 106 can exhibit an elongated shape that is configured to extend from a first location below the urethral opening (e.g., at or near the anus or the vaginal opening) to a second location above the urethral opening (e.g., at or near the clitoris or the pubic hair). The opening 106 can exhibit an elongated shape since the space between the legs of a female is relatively small when the legs of the female are closed thereby only permitting the flow of the fluids along a path that corresponds to the elongated shape of the opening 106. The opening 106 can exhibit a width that is measured transverse to the longitudinal direction that is at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. In some embodiments, the opening 106 may be vertically oriented (e.g., having a major axis that is generally parallel to the longitudinal axis of the device 100). In some embodiments, (not shown), the opening 106 may be horizontally oriented (e.g., having a major axis perpendicular to the longitudinal axis of the device 100). In an example, the fluid impermeable barrier 102 can be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 is also configured to temporarily store the fluids in the chamber 104. For example, the fluid impermeable barrier 102 can be formed of any suitable fluid impermeable materials, such as a fluid impermeable polymer (e.g., silicone, polypropylene, polyethylene, polyethylene terephthalate, a polycarbonate, etc.), a metal film, another suitable material, or combinations thereof. As such, the fluid impermeable barrier 102 substantially prevents the fluids from exiting the portions of the chamber 104 that are spaced from the opening 106. In an example, the fluid impermeable barrier 102 can be air permeable and fluid impermeable. In such an example, the fluid impermeable barrier 102 can be formed of a hydrophobic material that defines a plurality of pores. In an example, at least one or more portions of an outer surface of the fluid impermeable barrier 102 can be formed from a soft and/or smooth material thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In an example, the markings may include one or more of alignment guide or an orientation indicator, such as a stripe or hashes. Such markings may be positioned to align the device 100 to one or more anatomical features, such as a pubic bone.

The fluid collection device 100 can include a fluid permeable membrane 120 disposed in the chamber 104. The fluid permeable membrane 120 can cover at least a portion (e.g., all) of the opening 106. The fluid permeable membrane 120 can be configured to wick any fluid away from the opening 106 thereby preventing the fluid from escaping the chamber 104. The permeable properties can have wicking, capillary action, diffusion, or other similar properties or processes, and are referred to herein as “permeable” and/or “wicking.” Such “wicking” may not include absorption into the wicking material. The fluid permeable membrane 120 can also wick the fluid generally towards an interior of the chamber 104. The fluid permeable membrane 120 can include any material that can wick the fluid. For example, the fluid permeable membrane 120 can include fabric, such as a gauze (e.g., a silk, linen, or cotton gauze), another soft fabric, or another smooth fabric. Forming the fluid permeable membrane 120 from gauze, soft fabric, and/or smooth fabric can reduce chaffing caused by the fluid collection device 100.

The fluid collection device 100 can include a fluid permeable support 122 disposed in the chamber 104. The fluid permeable support 122 is configured to support the fluid permeable membrane 120 since the fluid permeable membrane 120 can be formed from a foldable, flimsy, or otherwise easily deformable material. For example, the fluid permeable support 122 can be positioned such that the fluid permeable membrane 120 is disposed between the fluid permeable support 122 and the fluid impermeable barrier 102. As such, the fluid permeable support 122 can support and maintain the position of the fluid permeable membrane 120. The fluid permeable support 122 can be formed from any fluid permeable material that is less deformable than the fluid permeable membrane 120. For example, the fluid permeable support 122 can include spun plastic fibers. In an example, the fluid permeable support 122 can be omitted from the fluid collection device 100.

In an embodiment, the fluid permeable membrane 120 and the fluid permeable support 122 can at least substantially completely fill the portions of the chamber 104 that are not occupied by the conduit 108. In an embodiment, the fluid permeable membrane 120 and the fluid permeable support 122 does not substantially completely fill the portions of the chamber 104 that are not occupied by the conduit 108. In such an embodiment, the fluid collection device 100 includes a reservoir 124 disposed in the chamber 104. The reservoir 124 is a substantially unoccupied portion of the chamber 104. The fluids that are in the chamber 104 can flow through the fluid permeable membrane 120 and/or fluid permeable support 122 to the reservoir 124. The reservoir 124 can store at least some of the fluids therein.

In an example, the reservoir 124 can be located at the end of the chamber 104 that is closest to the aperture 110. However, the reservoir 124 can be located at different locations in the chamber 104. For example, the reservoir 124 can be located at the first end of the chamber 104 that is closest to the outlet 128. In another example, fluid collection device 100 can include multiple reservoirs, such as a first reservoir that is located at the end of the chamber 104 that is closest to the aperture 110 and a second reservoir that is located at the end of the chamber 104 that is closest to the outlet 128. In another example, the fluid permeable support 122 is spaced from at least a portion of the conduit 108 and the reservoir 124 can be the space between the fluid permeable support 122 and the conduit 108. In some embodiments (not shown), the conduit 108 may enter the second end and the at least one aperture 110 of the conduit 108 may be disposed in the second end region (e.g., in the reservoir 124). In such examples, the first end may be substantially sealed.

Other examples of fluid impermeable barriers, fluid permeable membranes, fluid permeable supports, and chambers are disclosed in U.S. patent application Ser. No. 15/260,103 filed on Sep. 8, 2016, the disclosure of which is incorporated herein, in its entirety, by this reference.

The conduit 108 includes and extends between at least one aperture 110 and an outlet 128 that is couplable to the valve 114 (e.g., attachable and/or in fluid communication with the first aperture 116). The aperture 110 allows the interior 112 of the conduit 108 to be in fluid communication with the chamber 104. For example, the aperture 110 allows a fluid to flow from the chamber 104 to the interior 112 of the conduit 108. In an embodiment, the aperture 110 can be disposed in or adjacent to the reservoir 124 thereby preventing pooling of the fluids in the reservoir 124. In an embodiment, the aperture 110 can be located at or near a gravimetrically low point of the fluid collection device 100 thereby preventing pooling of the fluids at the gravimetrically low point of the fluid collection device 100. In an embodiment, the aperture 110 can be spaced from the reservoir 124, such as adjacent to the fluid permeable membrane 120 and/or the fluid permeable support 122 thereby preventing fluid stagnation in the fluid permeable membrane 120 and/or the fluid permeable support 122. In an embodiment, as shown, the conduit 108 only includes a single aperture 110. In another embodiment, the conduit 108 can include a plurality of apertures. In such an embodiment, the conduit 108 can include at least one aperture disposed in or near the reservoir 124 and, optionally, at least one aperture that is spaced from the reservoir 124. In some embodiments, the one or more portions of the conduit 108 and/or at least one tube connected to the conduit 108 is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety. As previously discussed, the valve 114 is spaced from the chamber 104. As such, the conduit 108 includes a first portion 130 that is disposed in the chamber 104 and a second portion 132 that is spaced from the chamber 104. For example, the second portion 132 can extend from the fluid impermeable barrier 102 to the valve 114. The fluid impermeable barrier 102 can define a hole 134 that allows the conduit 108 to extend therethrough. The first portion 130 of the conduit 108 can define the aperture 110 and the second portion 132 can define the outlet 128.

As previously discussed, the valve 114 includes a first aperture 116 and a second aperture 118. The first aperture 116 is configured to be coupled to the outlet 128 of the conduit 108. The second aperture 118 is configured to be coupled to at least one of the vacuum source or the fluid storage container. In an example, the second aperture 118 is indirectly coupled to the vacuum source or the fluid storage container via at least one tube 136.

The fluid collection device 100 also includes at least one actuator 138 that is coupled to or integrally formed with the valve 114. The actuator 138 is configured to be manipulated by a user of the fluid collection device 100. Manipulating the actuator 138 by the individual can cause actuation of the valve 114 (e.g., switch the valve 114 between the first state and the second state). For example, manipulating the actuator may activate the actuator 138 which, in turn, controllably actuates the valve 114. It is noted that the valve 114 can include at least one intermediate state that allows more gas to flow through the conduit 108 than when the valve 114 is in the first state and less gas to flow through the conduit 108 than when the valve 114 is in the second state and manipulating the actuator 138 can cause the valve 114 to controllably exhibit the intermediate state.

In an embodiment, as shown, the actuator 138 is a lever, a handle, a handwheel, or another suitable mechanical device that can be manipulated (e.g., manually manipulated) by the individual and is coupled to (e.g., disposed on or attached to) or integrally formed with the valve 114. In such an embodiment manipulating the actuator 138 can create a mechanical force which switches the valve 114 between the first and second states. In an embodiment, the actuator 138 can include a button or electrical switch (e.g., an electrical switch in the form of a lever) that can be manipulated by the individual and an electric motor, hydraulic device, pneumatic device, etc. (collectively referred to as the “machine actuator”) that is activated by manipulating the button or electrical switch. Manipulating the button or electrical switch can cause the machine actuator to switch the valve 114 between the first and second states. In such an embodiment, the at least a portion of the actuator 138 can be coupled to the valve 114 and/or at least a portion of the actuator 138 can be spaced from the valve 114. For example, the button or electrical switch can be spaced from the valve 114 while the machine actuator is disposed on, attached to (e.g., via a belt or chain), or integrally formed with the valve 114. In such an example, the button or electrical switch and machine actuator can each include a transmitter (not shown) which allows the button or electrical switch to transmit electrical signals to the machine actuator instructing the machine actuator to switch the valve 114 between the first and second states responsive to direction from the individual.

The valve 114 can include any suitable valve. For example, the valve 114 can include a ball valve, a butterfly valve, a ceramic disc valve, a check valve, a choke valve, a diaphragm valve, a gate valve, a globe valve, a knife valve, a needle valve, or a pinch valve. It is noted that the type of valve can depend on several factors, such as the type of actuator 138 of the valve 114, whether the actuator 138 is configured to generate a force or electrical signal that switches the valve 114 between the first and second states, whether the valve 114 includes one or more intermediate states, the requisite size of the valve 114, etc.

In an example, one or more components of the fluid collection device 100 can include an antimicrobial material, such as an antibacterial material on any surface of the fluid collection device that may contact the wearer or the bodily fluid of the wearer. The antimicrobial material can include an antimicrobial coating, such as a nitrofurazone or silver coating. The antimicrobial material can inhibit microbial growth, such as microbial growth due to pooling or stagnation of the bodily fluids. In an example, one or more components (e.g., impermeable barrier 102, conduit 108, etc.) of the fluid collection device 100 can include an odor blocking or absorbing material such as a cyclodextrine containing material or a thermoplastic elastomer (TPE) polymer.

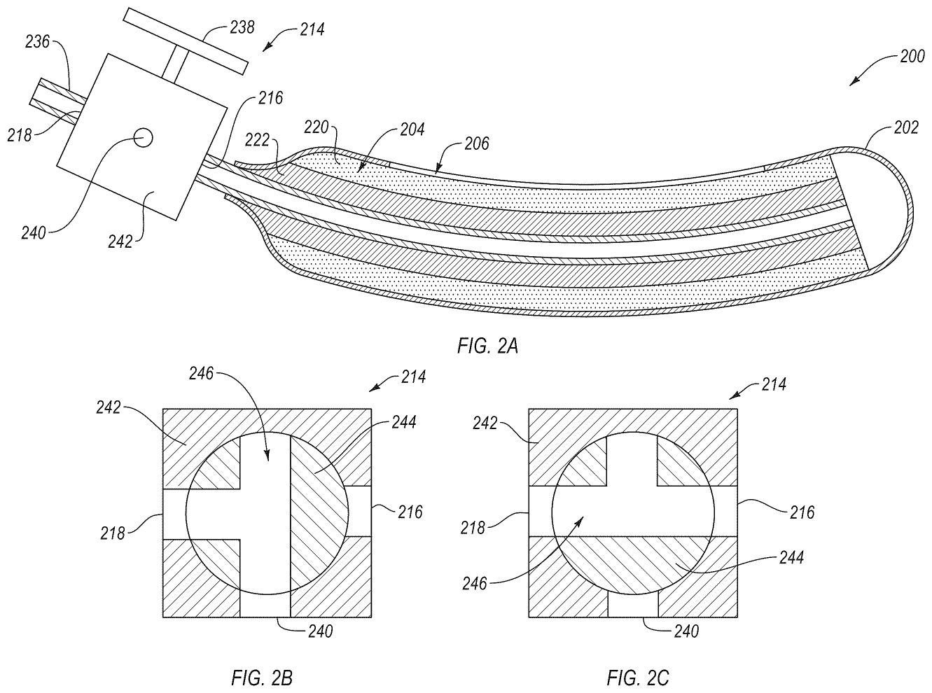

FIG. 2A is a schematic cross-sectional view of a fluid collection device 200, according to an embodiment. Except as otherwise disclosed herein, the fluid collection device 200 can be the same as or substantially similar to any of the fluid collection devices disclosed herein. For example, the fluid collection device 200 can include a fluid impermeable barrier 202 that defines a chamber 204 and an opening 206. The fluid collection device 200 can also include a fluid permeable membrane 220 and a fluid permeable support 222 disposed in the chamber 204. The fluid collection device 200 also includes a valve 214 and a conduit 208 that allows the valve 214 to be in fluid communication with the chamber 204.

The valve 214 includes a first aperture 216 that is couplable to the conduit 208 and a second aperture 218 that is couplable to at least one of a vacuum source or a fluid storage container. The second aperture 218 can be, for example, indirectly coupled to at least one of the vacuum source or the fluid storage container with a tube 236. The fluid collection device 200 also includes an actuator 238 that can be manipulated by an individual using the fluid collection device 200 to selectively and controllably switch the valve 214 between the first and second states.

The valve 214 further includes at least one bleeder outlet 240 that is configured to enable a gas flow through the valve 214 (but not the conduit 208) when the valve 214 is in the first state. For example, the bleeder outlet 240 is configured to enable gas flow between the bleeder outlet 240 and the second aperture 218 of the valve 214 when the gas flow through the conduit 208 is partially and/or at least substantially completely restricted by the valve 214 (e.g., the valve 214 is in the first state). As such, the valve 214 is at least a three way valve (e.g., the valve 214 is a three way valve when the valve 214 includes a single bleeder outlet 240, a four way valve when the valve 214 includes two bleeder outlets 240, etc.). In an example, enabling the gas flow through the valve 214 when the valve 214 is in the first state can prevent a vacuum force from crimping the tube 236 when the second aperture 218 is coupled to a vacuum source. Crimping the tube 236 can impede gas and fluid flow through the tube 236 when the valve 214 is in the first state and/or cause damage to the tube 236 which can impede the gas and fluid flow through the tube 236 when the valve 214 is in the second state. In another example, enabling the gas flow through the valve 214 when the valve 214 is in the first state can prevent a pressure buildup in the tube 236 when the second aperture 218 is coupled to a gas source. The buildup of pressure can cause the tube 236 to rupture or otherwise deform.

FIGS. 2B and 2C are schematic cross-sectional views of the valve 214 in a first and second states, respectively, according to an embodiment. The valve 214 shown in FIGS. 2B-2C is a three way ball valve. However, it is understood that the valve can include other types of valves, such as a four way ball valve, a butterfly valve, etc. The valve 214 includes a body 242 and a ball 244. The body 242 defines the first aperture 216, the second aperture 218, and the bleeder outlet 240. The body 242 also defines a hollow portion that includes the ball 244 moveably disposed therein. The ball 244 defines a passageway 246 therein. The passageway 246 can exhibit a generally straight path, a generally L-shape path, a generally T-shaped path, or any other suitable path. FIG. 2B illustrates the position of the ball 244 when the valve 214 is in the first state. For example, when the valve 214 is in the first state, the ball 244 is positioned such that the passageway 246 is in fluid communication with the second aperture 218 and the bleeder outlet 240. As such, the bleeder outlet 240 allows gas to flow through the valve 214 but not the conduit 208. FIG. 2C illustrates the position of the ball 244 when the valve 214 is in the second state. For example, when the valve 214 is in the second state, the ball 244 is positioned such that the passageway 246 is in fluid communication with the first and second apertures 216, 218 but not the bleeder outlet 240. As such, the gas flows through the conduit 208.

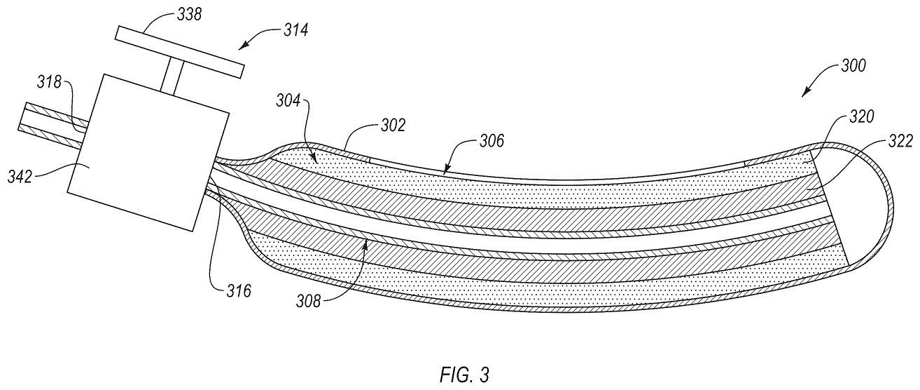

FIG. 3 is a schematic cross-sectional view of a fluid collection device 300, according to an embodiment. Except as otherwise disclosed herein, the fluid collection device 300 is the same as or substantially similar to any of the fluid collection devices disclosed herein. For example, the fluid collection device 300 can include a fluid impermeable barrier 302 that defines a chamber 304 and an opening 306. The fluid collection device 300 can also include a fluid permeable membrane 320 and a fluid permeable support 322 disposed in the chamber 304. The fluid collection device 300 also includes a valve 314 and a conduit 308 that allows the valve 314 to be in fluid communication with the chamber 304. The valve 314 includes a first aperture 316 and a second aperture 318 and. The fluid collection device 300 further includes an actuator 338.

The valve 314 abuts the fluid impermeable barrier 302 but is not disposed in the chamber 304. Positioning the valve 314 to abut the fluid impermeable barrier 302 can decrease the overall size of the fluid collection device 300. For example, positioning the valve 314 to abut the fluid impermeable barrier 302 can cause the conduit 308 to be completely positioned in the chamber 304 thereby eliminating the need for a second portion (e.g., second portion 132 of FIG. 1) of the conduit 308. Decreasing the size of the fluid collection device 300 makes it easier to hide the presence of the fluid collection device 300, such as hide the fluid collection device 300 under clothing (e.g., under the under garments of the individual).

In an embodiment, the valve 314 is distinct from the fluid impermeable barrier 302. In an embodiment, the valve 314 is at least partially integrally formed with the fluid impermeable barrier 302. In such an embodiment, the body 342 of the valve 314 is formed from the same material as the fluid impermeable barrier 302. In an embodiment, the valve 314 at least partially defines the chamber 304.

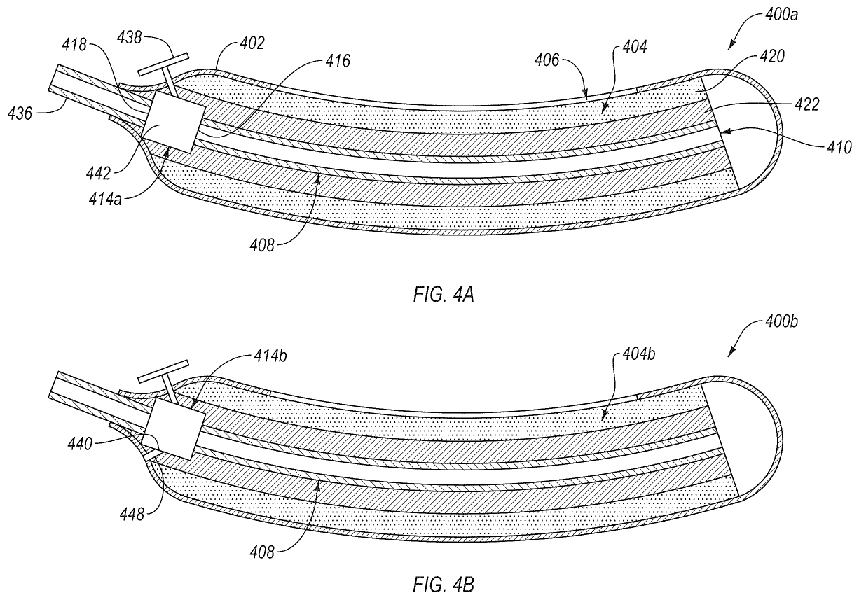

FIG. 4A is a schematic cross-sectional view of a fluid collection device 400a, according to an embodiment. Except as otherwise disclosed herein, the fluid collection device 400a is the same as or substantially similar to any of the fluid collection devices disclosed herein. For example, the fluid collection device 400a can include a fluid impermeable barrier 402 that defines a chamber 404a and an opening 406. The fluid collection device 400a can also include a fluid permeable membrane 420 and a fluid permeable support 422 disposed in the chamber 404a. The fluid collection device 400a also includes a valve 414a and a conduit 408 that allows the valve 414a to be in fluid communication, indirectly, with the chamber 404a. The valve 414a includes a first aperture 416 and a second aperture 418. Further, the fluid collection device 400a includes an actuator 438.

The valve 414a is at least partially disposed in the chamber 404a. For example, the body 442 of the valve 414a is completely disposed in the chamber 404a while the actuator 438 of the valve 414a extends from the body 442 to location that is spaced from the chamber 404a. As such, the actuator 438 is positioned in a location that an individual using the fluid collection device 400a can easily access. Since the valve 414a is at least partially disposed in the chamber 404a, a tube 436 may need to be inserted into the chamber 404a.

Since the valve 414a is disposed in the chamber 404a, the valve 414 is exposed to the fluids. As such, the valve 414a can be formed from materials that can be exposed to the fluids without being damaged (e.g., rusted, corroded, etc.). For example, the valve 414a (e.g., the body 442 of the valve 414a) can be formed from stainless steel, copper or copper alloys, graphite, aluminosilicates, silica, zinc plated steel, plastic (e.g., polyfluoroethylene resin, or polyethylene, polystyrene), or other suitable material. In an example, the body 442 can form a substantially fluid tight seal with the conduit 408, the tube 436, and the actuator 438 such that the fluids do not enter one or more interior portions of the valve 414 that are not configured to receive the fluids since fluids entering these portions of the valve 114 can make it harder to operate the valve 414a.

At least partially positioning the valve 414a in the chamber 404a can decrease the overall size of the fluid collection device 400a. Additionally, at least partially positioning the valve 414a in the chamber 404a can reduce pooling and/or stagnation of the fluids in the chamber 404a. For example, the presence of the valve 414a decreases the volume of the chamber 404a that can hold the fluids. While the valve 414a decreases the amount of fluids that the fluid collection device 400a can hold, it also causes more of the fluid to flow towards the reservoir 424 and/or the aperture 410 of the conduit 408 thereby reducing pooling or stagnation of the fluids in the chamber 404a.

FIG. 4B is a schematic cross-sectional view of a fluid collection device 400b, according to an embodiment. The fluid collection device 400b is the same as the fluid collection device 400a of FIG. 4A except that the valve 414b includes a bleeder outlet 440. As such, the valve 414b can include a bleeder tube 448 that extends from the bleeder outlet 440b to a location that is spaced from the chamber 404b. Without the bleeder tube 448, the fluid collection device 400b would continue to cause discomfort to an individual using the fluid collection device 400b even when the valve 414b is in the first state.

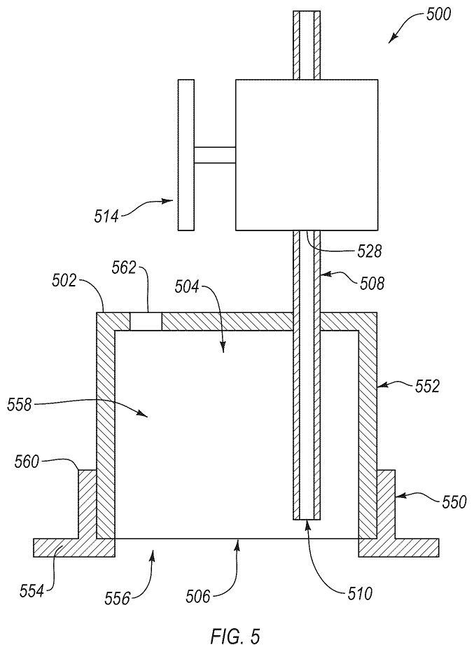

The fluid collection devices shown in FIGS. 1-4B are examples of female fluid collection devices that are configured to collect fluids from females (e.g., collect urine from a female urethra). However, any of the fluid collection devices disclosed herein can be male fluid collection devices that are configure to collect fluids from males (e.g., collect urine from a male urethra). FIG. 5 is a schematic cross-sectional view of male fluid collection device 500, according to an embodiment.

The fluid collection device 500 includes a receptacle 550 and a cup portion 552. The receptacle 550 is configured to be coupled to skin that surrounds the male urethra and have the male urethra positioned therethrough. For example, the receptacle 550 can include an annular base 554 that defines a hole 556. The annular base 554 is configured to be positioned around the male urethra (e.g., positioned around the penis) and the hole 556 can be configured to have the male urethra positioned therethrough. The annular base 554 can also be configured to be coupled (e.g., adhesively attached, such as with a hydrogel adhesive) to the skin around the male urethra. In an example, the annular base 554 can exhibit the general shape of the skin surface that the annular base 554 is configured to be coupled and/or can be flexible thereby allowing the annular base 554 to conform to the shape of the skin surface. The receptacle 550 also defines a hollowed region 558 that is configured to have the cup portion 552 disposed therein. For example, the receptacle 550 can include a flange 560 that extends upwardly from the annular base 554 that partially defines the hollowed region 558. The hollowed region 558 is deep enough that the cup portion 552 is unlikely to be accidentally removed from the hollowed region 558 (e.g., the hollowed region 558 is at least 1 cm deep, at least 2 cm deep, or at least 5 cm deep).

The cup portion 552 includes a fluid impermeable barrier 502 that is sized and shaped to fit into the hollowed region 558 of the receptacle 550. The fluid impermeable barrier 502 partially defines a chamber 504. The fluid impermeable barrier 502 also defines an opening 506 extending through the fluid impermeable barrier 502 that is configured to have a male urethra positioned therethrough. The fluid impermeable barrier 502 can also define at least one passthrough 562 that allows the chamber 504 to remain substantially at atmospheric pressure. The cup portion 552 also include a conduit 508 that is at least partially disposed in the chamber 504. The conduit 508 includes an aperture 510 that allows an interior 512 of the conduit 508 to be in fluid communication with the chamber 504. The conduit 508 also includes an outlet 528 that is configured to be communicably coupled to a valve 514.

In an example, the chamber 504 can be substantially empty due to the varying sizes and rigidity of the male penis. However, the outermost regions of the chamber 504 can include a porous material (not shown) configured to blunt a stream of urine from the male urethra thereby limiting splashing and/or to direct the fluids to a selected region of the chamber 504. Since the chamber 504 is substantially empty (e.g., substantially all of the chamber 504 forms a reservoir), the fluids are likely to pool at a gravimetrically low point of the chamber 504. The gravimetrically low point of the chamber 504 can be at an intersection of the skin of an individual and the fluid collection device 500, a corner formed in the cup portion 552, or another suitable location. The aperture 510 of the conduit 508 can be configured and positioned to be adjacent or proximate to the gravimetrically low point of the chamber 504. In an example, the chamber 504 may include at least one of a fluid permeable membrane (not shown) or support (not shown) disposed therein that is similar to the fluid permeable membranes and supports, respectively, disclosed herein.

The fluid collection device 500 also includes a valve 514. The valve 514 can include any of the valves disclosed herein. In an example, as illustrated, the valve 514 can be spaced from the chamber 504. However, in other examples, the valve 514 can abut the fluid impermeable barrier 502 or can be at least partially disposed in the chamber 504.

FIG. 6 is a schematic illustration of a fluid collection system 664, according to an embodiment. The system 664 includes a fluid collection device 600. The fluid collection device 600 can be the same as or substantially similar to any of the fluid collection devices 100, 200, 300, 400a, 400b, or 500 of FIGS. 1-5. The system 664 also include a fluid storage container 668 that is in indirect fluid communication with the fluid collection device 600 via a first tube 636. The system 664 also includes a vacuum source 670 that is in indirect fluid communication with to the fluid storage container 668 via a second tube 672. As such, the vacuum source 670 is in indirect fluid communication with the fluid collection device 600 via the first and second tubes 636, 672 and the fluid storage container 668. During operation, the vacuum source 670 can supply a vacuum to the fluid collection device 600. When the valve (not shown) of the fluid collection device 600 is in the second state, the vacuum pulls fluids from the fluid collection device 600 and deposits the fluids in the fluid storage container 668.

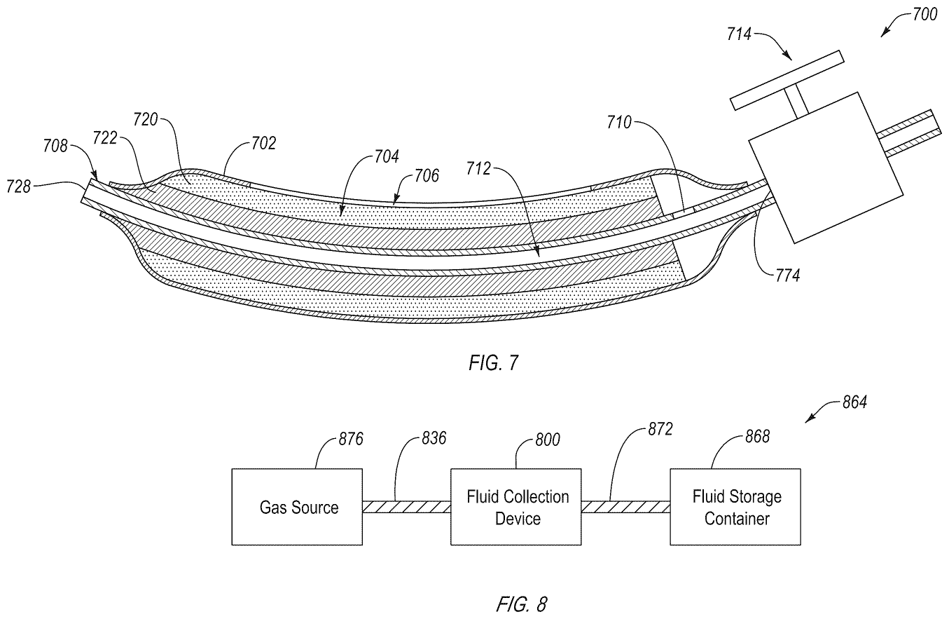

The fluid collection devices 100, 200, 300, 400a, 400b, 500, and 600 of FIGS. 1-6 are configured to be coupled to a vacuum source. However, any of the fluid collection devices disclosed herein can be configured to be coupled to a gas source, such as a pump, a compressor, or a tank of compressed gas. FIG. 7 is a schematic cross-sectional view of a fluid collection device 700 that is configured to be coupled to a gas source, according to an embodiment. Except as otherwise disclosed herein, the fluid collection device 700 is the same as or substantially similar to any of the fluid collection devices disclosed herein. For example, the fluid collection device 700 includes a fluid impermeable barrier 702 that defines a chamber 704 and an opening 706. The fluid collection device 700 can include a fluid permeable membrane 720 and a fluid permeable support 722 when the fluid collection device 700 is configured to collect fluids from a female. The fluid collection device 700 also includes a conduit 708 that is at least partially disposed in the chamber 704 and a valve 714. In an embodiment, as illustrated, the valve 714 is spaced from the fluid impermeable barrier 702 and the chamber 704. However, in other embodiments, the valve 714 can abut the fluid impermeable barrier 702 and/or be at least partially disposed in the chamber 704.

The conduit 708 includes and extends between an inlet 774 and an outlet 728. The inlet 774 is configured to be indirectly coupled to a gas source (e.g., gas source 876 of FIG. 8) and the outlet 728 is configured to be directly or indirectly coupled to a fluid storage container (e.g., fluid storage container 868 of FIG. 8). As such, the gas flow through the interior 712 of the conduit 708 flows from the inlet 774 to the outlet 728. The conduit 708 can include at least one aperture 710 that allows the interior 712 of the conduit 708 to be in fluid communication with the chamber 704. The valve 714 is positioned at a location that is upstream from the aperture 710 thereby allowing the valve 714 to regulate the gas flow before the gas reaches the aperture 710.

In an embodiment, the conduit 708 is configured to not use suction to remove fluids from the chamber 704. In such an embodiment, the conduit 708 substantially only influences the flow of the fluid when the fluid enters the conduit 708. In an embodiment, the conduit 708 is configured to use suction to remove fluids from the chamber 704. In such an embodiment, the conduit 708 can include a suction device that is configured to apply a suction force to the chamber 704. Examples of suction devices that can actively remove fluids from the chamber 704, along with additional examples of fluid collection devices that are configured to be coupled to a gas source, are disclosed in U.S. Provisional Patent Application No. 62/665,331 filed on 1 May 2018, the disclosure of which is incorporated herein, in its entirety, by this reference.

FIG. 8 is a schematic of a fluid collection system 864, according to an embodiment. The system 864 includes a fluid collection device 800. The system 864 also includes a gas source 876 that is positioned upstream from the fluid collection device 800 and is coupled to the valve (not shown) the fluid collection device 800. For example, the gas source 876 can be indirectly coupled to the valve via at least one first tube 836. The system 864 also includes a fluid storage container 868 positioned downstream from the fluid collection device 800. The fluid storage container 668 can be in fluid communication with the outlet of the conduit, such as indirect fluid communication with the outlet via a second tube 872.

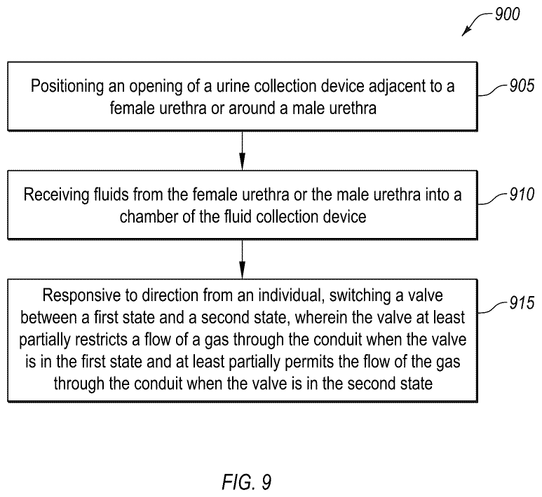

FIG. 9 is a flow diagram of a method 900 to use any of the fluid collection devices and/or fluid collection systems disclosed herein, according to an embodiment. The method 900 can include act 905, which recites “positioning an opening of a urine collection device adjacent to a female urethra or around a male urethra.” Act 905 may be followed by act 910, which recites “receiving fluids from the female urethra or the male urethra into a chamber of the fluid collection device.” Act 910 may be followed by act 915, which recites “responsive to direction from an individual, switching a valve between a first state and a second state, wherein the valve at least partially restricts a flow of a gas through the conduit when the valve is in the first state and at least partially permits the flow of the gas through the conduit when the valve is in the second state.”

Acts 905, 910, and 915 of the method 900 are for illustrative purposes. For example, the act 905, 910, and 915 of the method 900 can be performed in different orders, split into multiple acts, modified, supplemented, or combined. In an example, one or more of the acts 905, 910, 915 of the method 900 can be omitted from the method 900.

Act 905 recites “positioning an opening of a fluid collection device adjacent to a female urethra or around a male urethra.” In an example, act 905 can include positioning the opening of a female fluid collection device such that the fluid permeable membrane of the female fluid collection device abuts or is positioned proximate to the female urethra. In another example, act 905 can include positioned a receptacle of a male fluid collection device around the male urethra such that the male urethra is positioned through a hole of the receptacle. In such an example, act 905 can include positioning a cup portion of the male fluid collection device in a hollowed region defined by the receptacle such that the male urethra is positioned through an opening of the cup portion.

Act 910 recites “receiving fluids from the female urethra or the male urethra into a chamber of the fluid collection device.” For example, act 910 can include wicking the fluids away from the opening using a fluid permeable membrane and a fluid permeable support. In another example, act 910 can include receiving the fluids into the chamber of the cup portion of the male fluid collection device. In either example, act 910 can include flowing the fluid towards a portion of the chamber that is in fluid communication with an aperture of a conduit. For instance, act 910 can include flowing the fluids to a substantially unoccupied portion of the chamber (e.g., a reservoir), to a gravimetrically low point of the chamber, etc.

Act 915 recites “responsive to direction from an individual, switching a valve between a first state and a second state, wherein the valve at least partially restricts a flow of a gas through the conduit when the valve is in the first state and at least partially permits the flow of the gas through the conduit when the valve is in the second state.” For example, act 915 can include switching the valve between the first and second states by manually manipulating an actuator disposed on the valve, such as twisting a handle, a wheel, or another suitable actuator. In another example, act 915 can include switching the valve between the first and second states by electronically manipulating an actuator that is disposed on or spaced from the valve, such as by pressing a button or electronic switch which then activates a motor, a pneumatic device, a hydraulic device, etc. In another example, act 915 can include flowing a gas through a bleeder outlet when the valve is in the first state.

In an example, act 915 can include switching a valve that is spaced from the chamber and the fluid impermeable barrier of the fluid collection device between the first and second state. In an example, act 915 can include switching a valve that abuts the fluid impermeable barrier between the first and second states. In an example, act 915 can include switching a valve that is at least partially disposed in the chamber between the first and second states.

While various aspects and embodiments have been disclosed herein, other aspects and embodiments are contemplated. The various aspects and embodiment disclosed herein are for purposes of illustration and are not intended to be limiting.

1. (canceled)

2. A fluid collection system, comprising:
a fluid collection device, comprising:
a fluid impermeable barrier defining at least a chamber, an opening, and an outlet, the fluid impermeable barrier also defining an opening extending therethrough; and
a single conduit coupled to the outlet, the conduit defining at least one aperture therein that allow an interior of the conduit to be in fluid communication with the chamber; and
a valve including a first aperture coupled to the single conduit and a second aperture, the valve configured to selectively control flow of a bodily fluid out of the chamber responsive to actuation of the valve, wherein the valve is a two-way valve.

3. The fluid collection system of claim 2, wherein the single conduit extends through the outlet and a portion of the single conduit is disposed in the chamber.

4. The fluid collection system of claim 2, wherein the valve is configured to switch between at least a first state and a second state responsive to the actuation of the valve, wherein gas flow from the first aperture to the second aperture is substantially completely restricted when in the first state and gas flow from the first aperture to the second aperture is permitted when in the second state.

5. The fluid collection system of claim 4, wherein the valve is configured to switch between the first state, the second state, and at least one intermediate state;
wherein, when in the at least one intermediate state, gas flow from the first aperture to the second aperture is greater and less than when the valve is in the first state and the second state, respectively.

6. The fluid collection system of claim 2, wherein the valve is configured to increase or decrease gas flow between the first aperture and the second aperture.

7. The fluid collection system of claim 2, wherein the valve is at least partially disposed in the chamber.

8. The fluid collection system of claim 2, wherein the valve abuts the fluid impermeable barrier.

9. The fluid collection system of claim 2, wherein the valve is spaced from the chamber.

10. The fluid collection system of claim 9, wherein the valve is spaced from the fluid collection device.

11. The fluid collection system of claim 2, wherein the fluid collection device further includes a fluid permeable outer layer and a fluid permeable inner layer positioned within the chamber.

12. The fluid collection system of claim 11, wherein the fluid permeable inner layer defines a bore configured to receive the conduit.

13. The fluid collection system of claim 11, wherein the fluid permeable outer layer extends across the opening.

14. The fluid collection system of claim 11, wherein at least one of the fluid permeable outer layer or the fluid permeable inner layer defines a cutout configured to receive the valve, wherein the valve is at least partially positioned within the cutout.

15. The fluid collection system of claim 2, further comprising an actuator coupled to or integrally formed with the valve for manual actuation by a user, wherein manipulating the actuator selectively controls flow of the fluid through the single conduit and out of the chamber.

16. The fluid collection system of claim 15, wherein the valve is at least partially positioned within the chamber, the fluid impermeable barrier defines a hole configured to have the actuator extend therethrough.

17. The fluid collection system of claim 2, further comprising a vacuum source in fluid communication with the second aperture.

18. The fluid collection system of claim 17, further comprising a fluid storage container in fluid communication with the vacuum source and the chamber.

19. The fluid collection system of claim 18, wherein the fluid storage container includes an inlet and an outlet, the inlet in fluid communication with the chamber via at least the single conduit, the outlet in fluid communication with the vacuum source via at least one tube.

20. A fluid collection system, comprising:
a fluid collection device, comprising:
a fluid impermeable barrier defining at least a chamber, an opening, and an outlet, the fluid impermeable barrier also defining an opening extending therethrough, the opening configured to be positioned adjacent to a female urethra or have a male urethra positioned therethrough;
a fluid permeable outer layer and a fluid permeable inner layer positioned within the chamber; and
a single conduit attached to the outlet, the conduit defining at least one aperture therein that allow an interior of the conduit to be in fluid communication with the chamber;
a valve including a first aperture coupled to the single conduit and a second aperture, the valve configured to selectively increase or decrease flow of a bodily fluid out of the chamber responsive to actuation of the valve, wherein the valve is a two-way valve;
a vacuum source in fluid communication with the second aperture; and
a fluid storage container in fluid communication with the vacuum source and the chamber.

21. A fluid collection system, comprising:
a fluid collection device, comprising:
a fluid impermeable barrier defining at least a chamber, an opening, and an outlet, the fluid impermeable barrier also defining an opening extending therethrough, the opening configured to be positioned adjacent to a female urethra or have a male urethra positioned therethrough; and
a single conduit attached to the outlet, the conduit defining at least one aperture therein that allow an interior of the conduit to be in fluid communication with the chamber;
a valve including a first aperture coupled to the single conduit and a second aperture, the valve configured to selectively control flow of a bodily fluid out of the chamber responsive to actuation of the valve, wherein the valve is a two-way valve, the valve is configured to switch between a first state, a second state, and at least one intermediate state;
wherein gas flow from the first aperture to the second aperture is substantially completely restricted when in the first state and gas flow from the first aperture to the second aperture is permitted when in the second state;
wherein, when in the at least one intermediate state, gas flow from the first aperture to the second aperture is greater and less than when the valve is in the first state and the second state, respectively;
a vacuum source in fluid communication with the second aperture; and
a fluid storage container in fluid communication with the vacuum source and the chamber, the fluid storage container including an inlet and an outlet, the inlet in fluid communication with the chamber via at least the single conduit, the outlet in fluid communication with the vacuum source via at least one tube.
